(19) United States
(12) Patent Application Publication
(10) Pub. No.: US 2009/0299771 A1
(43) Pub. Date: Dec. 3, 2009

(54) DICOM-BASED 12-LEAD ECG GATEWAY AND BROWSER UNDER THE CLINICALLY-USED INFORMATION SYSTEM

(75) Inventors: Jui-Chiem Hsieh, TAOYAUN (TW); Kuo Chiang Yu, TAOYAUN (TW); Hsiu-Chiung Lo, TAOYAUN (TW); Chia Chang Hung, TAOYAUN (TW); Li-Chern Pan, TAOYAUN (TW); Po-Wei Lin, TAOYAUN (TW)

Correspondence Address: WPAT, PC;INTELLECTUAL PROPERTY ATTORNEYS, 2030 MAIN STREET, SUITE 1300, IRVINE, CA, 92614, US

(73) Assignee: Yuan Ze University, TAOYAUN (TW)

(21) Appl. No.: 12/474,088

(22) Filed: May 28, 2009

(30) Foreign Application Priority Data
May 30, 2008 (TW) 097120303

(51) Int. Cl. G06Q 50/00; A61B 5/04; G06F 19/00; G06F 17/30; G06F 3/01; G06F 15/16; G06F 17/00

(52) U.S. Cl. 705/3; 600/509; 702/19; 707/102; 715/733; 709/247; 715/234

(57) ABSTRACT

The present invention relates to a DICOM-based 12-lead ECG gateway and browser for use in clinical information system.

### FIELD OF THE INVENTION

The present invention relates to a clinical 12-lead ECG gateway that converts SCP-ECG or XML-ECG format to DICOM-ECG format and a browser that integrates Picture Archiving and Communication Systems (PACS) and Healthcare Information Systems (HIS).

### BACKGROUND OF THE INVENTION

The Digital Imaging and Communications in Medicine (DICOM) (3.0) has recently suggested the one-dimensional biomedical signal standard, for example, blood pressure and ECG. The Picture Archiving and Communication Systems (PACS), constructed based on the standard, is used in most hospitals; its functions include image capturing, transferring, storage and management.

The advantage of using DICOM as the ECG standard is that it can be integrated into the existing PACS of the medical image storage system in hospitals, enhancing the exchanges of digital ECG data. Electronic ECG diagnosis reports are expected to arise based on the DICOM standard to allow intercompatibility between difference models for barrier-free data exchange.

Traditionally, for 12-lead ECG instruments to perform functions such as read, diagnose or save requires paying ECG instrument manufacturers to use their ECG management information software. For example, the PHILIPS Tracemaster developed by PHILIPS, and the HP ECG Manager by HP. These technology could be found in Taiwan patents such as TW patent No. 363404 and TW patent No. 1289052. The main reason is, the output formats of the 12-lead ECG instruments currently used in hospitals, like the Standard Communications Protocol for Computer-Assisted Electrocardiography (SCP-ECG) and the Extensible Markup Language Electrocardiography (XML-ECG), do not have strictly defined standard, allowing manufacturing companies to conceal or set up their own standards for the compression of ECG signals. Only those hospitals with sound financial standing could afford to purchase systems or hardware facilities of specific brands or models and own information of electronic ECG diagnosis reports, but these data still cannot be integrated with the Healthcare Information Systems.

Therefore, the conventional way in hospitals to generate an image file is to either have someone scan the ECG paper report or utilize image-yielding devices, in which a DICOM header is added by PACS manufacturers to enable browsing the ECG using PACS. Communication Gateway in line with the DICOM standard that converts the SCP-ECG directly to DICOM-ECG format, brought forward by foreign scholars recently, is capable of retrieving the waveforms from the raw data; however, it only works with standard SCP-ECG file formats and is unable to store the converted ECG diagnosis data; thus it still cannot integrate the file formats of 12-lead ECG instruments of all the leading companies, such as SCP-ECG by HP or XML-ECG by PHILIPS, and is not suitable for clinical use (V Sakkalis, F Chiarugi, S Kostomanolakis, C E Chronaki, M Tsiknakis, S C Orphanoudakis, “A gateway between the SCP-ECG and the DICOM supplement 30 waveform standard”, IEEE Computers in Cardiology, Vol 30, 25-28, 2003).

The DICOM browser that can show DICOM wave objects, together with the converter that converts the image files scanned from 12-Lead ECG paper reports to DICOM standard formats were suggested. The browser is based on the secondary retrieving module of DICOM standard, while the ECG wave data, using the format described as the 12-Lead wave module, is converted to the standard ECG images and wave forms of DICOM objects, which are stored together in the DICOM server (An ECG Image and Curve Display Environment in DICOM, Hsin-Yi Lin, Journal of Medical and Biological Engineering, Vol 24, 29-34, 2004). The idea of producing image files with image-yielding devices and converting them into standard DICOM file formats can be found in Taiwan Patents such as TW publication No. 200422908, 200423645, 200423740 and 200529019, and TW patent No. 1228375 and 1229281. However, this is not practical in clinical ECG diagnosis, because cost-inefficient scanning takes time and labor and big image files take up too much storage space and cost.

Although some international studies proposed the method of converting standard SPC-ECG to DICOM-ECG, they are limited to standard SCP-ECG files and cannot be applied to files of SCP-ECG by HP and of XML-ECG by PHILIPS in clinical use. An urgent task at the present stage is to integrate DICOM-ECG into HIS and PACS, so that clinical medical staff could schedule 12-Lead ECG examinations via HIS and review the data in high resolution, since the DICOM ECG converted from different formats are distortion-free.

Currently none of the clinical ECG instruments, by HP, PHILIPS or GE, used in hospitals can output DICOM-ECG format. The common practice is to convert the output to JPG or TIF image formats using the purchased software, and then store it in the PACS after adding a DICOM header. Nevertheless, the low-resolution stored images show distortions when they are zoomed in, and they are big in size and very costly. What makes people more astonished is that examinations still need to be scheduled on paper because the image file formats can not be integrated into HIS.

Previous studies also proposed methods of converting SCP-ECG and XML-ECG to DICOM 12-Lead ECG, but they are far from clinical application. These studies are:

### (1) Development of SCP to DICOM 12-Lead ECG Gate Server (I-Hsuan Chao, 2005);

(2) A Novel DICOM-Based ECG Documentary System (Hsieh Long Yi, 2006), which has the main purpose of converting collected SCP files to DICOM-ECG.; and
(3) A novel DICOM-Based 12-Lead ECG Documentary System (Hsieh J C, Journal of Electrocardiology, Vol 40:S81-S87. 2007). The clinical PHILIPS XML files are subject to noise interference so that a previous wave filter processing is needed. Furthermore, there are many things to be overcome, such as retrieving files from ECG instruments, integrating the files into clinical information system and linking transmission of the files.

There are still great obstacles to overcome before present 12-Lead ECG image formats are integrated into the HIS.

### BRIEF DESCRIPTION OF THE DRAWINGS

FIG. 1 is a system architecture diagram.

FIG. 2 is a flowchart of decoding HP SCP-ECG files.

FIG. 3 is a flowchart of decoding PHILIPS XML-ECG files.

FIG. 4 is a flowchart of converting SCP-ECG or XML-ECG to DICOM-ECG.

FIG. 5 shows the process of sketching clinical ECG from DICOM-ECG.

FIG. 6 shows the relationship between DICOM-ECG and HIS and PACS.

FIG. 7 shows the 12-Lead ECG scheduling information system.

FIG. 8 is a diagram of using the DICOM-ECG browser.

FIG. 9 shows the simultaneous diagnosis with other medical images while browsing ECG.

### SUMMARY OF THE INVENTION

The present invention relates to a DICOM-based 12-lead ECG gateway and browser for use in clinical information system.

### DETAILED DESCRIPTION OF THE INVENTION

The invention provides a data processing system of 12-Lead DICOM ECG that integrates with HIS or PACS, comprising: (a) a decoding and signal processing device that automatically retrieves SCP-ECG or XML-ECG used in a clinical ECG instrument; (b) a device that converts the decoded ECG signals to DICOM-ECG; (c) an ECG processing unit of 12-Lead DICOM ECG information management system, which receives signals from HIS or transmits DICOM-ECG to PACS; and (d) a 12-Lead DICOM-ECG browser that is switched on using HIS or PACS. The device for decoding SCP-ECG device in the system of the present invention comprises: (i) a device that processes the decoding of sections 0, 1, 8, 128; and (ii) a device that processes Huffman decoding. The device for decoding XML-ECG in the system of the present invention comprises: (iii) a device that reads report information (reportinfo tag), data acquisition information (measurements tag), patients' information (patient tag), interpretation information (interpretations tag) and waveforms information (waveforms tag); and (iv) a signal-processing device.

The ECG signals generated by stationary wavelet conversion using data obtained from decoding the SCP-ECG or XML-ECG include raw data of ECG waveforms or ECG diagnostic information; the latter includes but not limited to an accession number, name, gender, date of examination, time of examination, or ECG diagnosis result of individual patients.

The device that converts the decoded ECG signals to DICOM-ECG in the system of the present invention comprises the generation of a core file and an index file. The core files are subdivided into the format conversion of SCP-ECG core files and XML-ECG core files. The conversion of SCP-ECG core file formats sequentially comprises signal length extension, sequence order swapping, lead order swapping, signal length calculation and the writing of waveform lengths (or level lengths or waveform data). The conversion of XML-ECG core file formats sequentially comprises lead order swapping, signal length calculation, and the writing of waveform lengths (or level lengths or waveform data). The index file utilizes the ECG diagnosis information to write custom tags, patient information or retrieves time and date.

The 12-Lead DICOM-ECG information management system, which is the ECG processing unit of the present invention, is a device which stores the files in PACS and reports the completion of diagnosis and the communication with the information page to the HIS. PACS is used to integrate the DICOM files between medical institutes. The DICOM-ECG browser is linked to PACS or HIS system for remote operation, wherein the operation comprises creating new files, opening old files, saving files, printing, magnifying, shrinking or moving the files.

### EXAMPLE

The examples below are non-limiting and are merely representative of various aspects and features of the present invention.

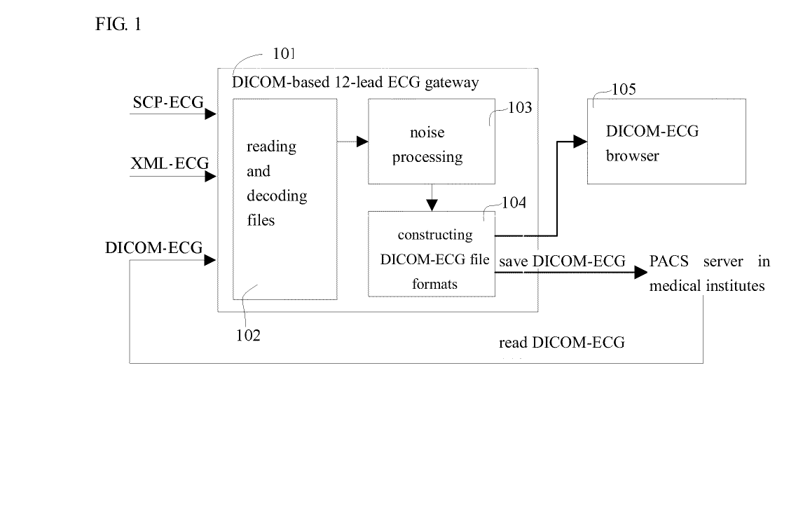

As shown in FIG. 1, the DICOM-based 12-lead ECG gateway 101 read and decoded the digital output files of HP-SCP ECG or PHILIPS-XML ECG from commonly used clinical 12-Lead ECG instruments 102; retrieved the ECG signals and computer-based automatic diagnosis of disease. Noise processing was proceeded with steady wave transformation 103, followed by constructing the DICOM-ECG file formats in accordance with international standards 104. Lastly, the ECG characteristics and ECG reports were observed and browsed through DICOM-ECG browser 105 or saved directly in the PACS server of respective medical institutes. Some of the preferred embodiments were detailed as follows.

### Example 1

### Processing SCP-ECG Files

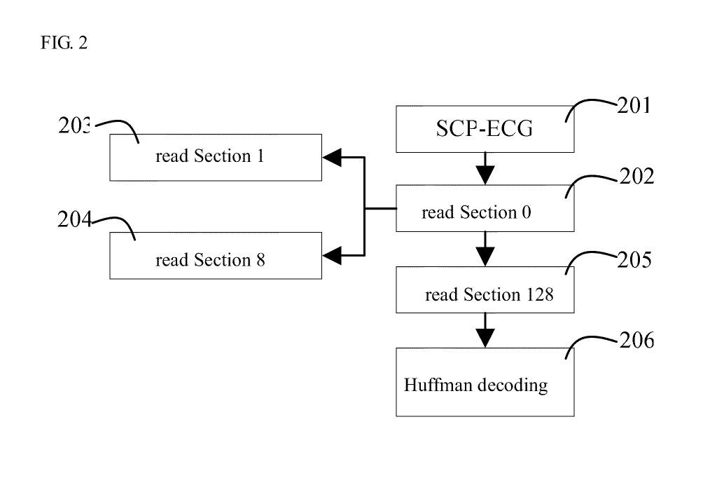

FIG. 2 showed the processing of digital output SCP-ECG files of the 12-Lead ECG instrument in the present invention. The clinically used SCP-ECG file formats were binary-based and consisted of many sections. The file processing of section 0, section 1, section 8 and section 128 was described as follows:
i. Reading the SCP-ECG file format 201 and subsequently reading the data in section 0 202. Section 0 was an index which referred to the location of all sections, in which the section with data was where the length and index value
of the section were non-zero. All sections consisted of 2-byte tags, 4-byte lengths and a 4-byte index, and each continuous byte was read from the high byte on the right to the low byte on the left. All the index values started from 1.
ii. Reading the data in section 1 203 where the patient information and ECG data were stored. The data were arranged in accordance with the method defined by standard SCP, and some additional customized tags were further added in.
iii. Reading the data in section 8 204 where the text results of ECG diagnosis and analysis were stored. Information was saved line by line to a total of 17 lines, comprising 2-byte tag (0D0A), 2-byte number of lines, 1-byte length, and a following part with data length of the lengths minus two, respectively. The texts were all stored in ASCII format.
iv. Reading the data in section 128 205 where the ECG signals were stored. There were 36 tags and 1 end tag in the ECG signals data and each of them had its unique code, which meant that once the first tag (1D01) was located, the location of the next tag could be inferred from the length of the first tag. Each component of ECG signals data consisted of 2-byte tags, 2-byte lengths and data blocks sorted by lengths. Every lead had a main tag and a sub tag, and the ECG signals were always stored under the main tag. There were a total of 12 short leads and 3 long leads, together with 6 unused tags and 1 end tag. At first, the first tag was accessed, followed by the 12 sets of main tags and sub tags (i.e. 12 short leads), and after skipping 3 tags, the 6 sets of main tags and sub tags (i.e. 3 long leads) were accessed. Lastly, the end tag was reached after skipping another 3 tags, and the ECG signals were retrieved.
v. Converting the ECG signals to binary format. Using Huffman tables, each lead was decoded. At the same time, decompression of continuous 0 was carried out, i.e. 3 bytes right after 9 continuous zeroes would be the storage length of 0.

### Example 2

### Processing XML-ECG Files

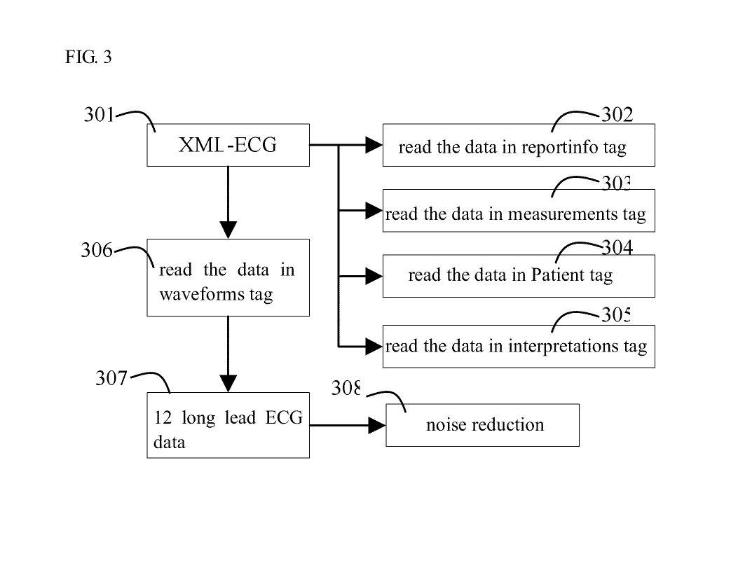

FIG. 3 showed the processing of digital output XML-ECG files of the 12-Lead ECG instrument in the present invention. The clinically used XML-ECG file format was based on ASCII. The processing of the files was described as follows:

Reading the XML-ECG file format 301. One by one, the data in the reportinfo tag 302, the measurement tag 303, the patient tag 304, the interpretation tag 305, and the waveform tag 306 were retrieved. After decoding using Base64, a total of 12 long-lead ECG signal data 307 were obtained. Lastly, noise reduction 308 was performed.

### Example 3

### Constructing DICOM-ECG 411

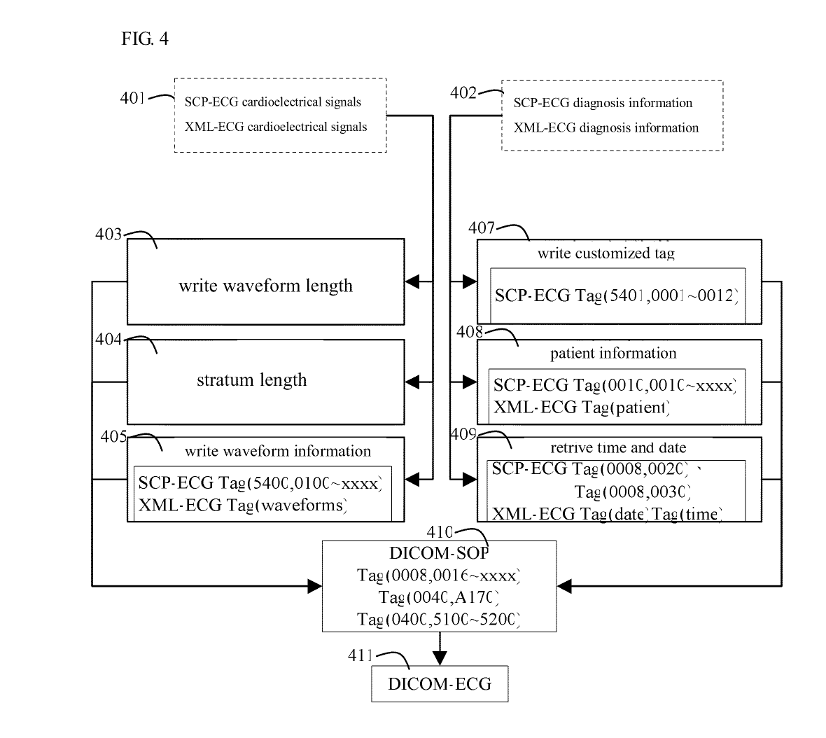

After the processing of the aforementioned SCP-ECG and XML-ECG files, the SCP-ECG and XML-ECG ECG signals were generated, including the raw data of 12-lead ECG waveforms and the ECG diagnosis data of the SCP-ECG and XML-ECG which consisted personal information of the patient, e.g. accession number, name, gender, date of examination, time of examination and the ECG diagnosis result. This present invention constructed DICOM-ECG file formats in accordance with international standards. The procedure, shown in FIG. 4, was illustrated as follows.

Writing the waveform data in the tag (5400, 1010) as performance value OW. OW (Other Word String) meant the data were written in 16-byte format. The waveforms contained all the data in the 12 leads, where they were arranged in the order of the sampling point of the 12 leads at the same time and written into the DICOM files according to this arrangement.

Modifying the waveform sequence (5400, 0100) and waveform data (5400, 1010) and defining the value length of the waveform sequence stratum (FFFE, E000) to make sure the integrity of the waveform data 404-405.

Writing the essential diagnosis information of the header in the DICOM file. Since there were no defined tags for ECG diagnosis information in the data dictionary of the DICOM document, customized tags were used to store data such as heartbeat, PR width, QRSD width, QT width, QTc, and the axis of P wave, QRS wave and T wave. The computer diagnosis information was written in the DICOM files and the information patterns of customized tags were defined by the numbers in the range of 0 to 216 of US (Unsigned Short) or LO. All of the above were done to maintain the integrity of the ECG diagnosis information 407-411.

### Example 4

### DICOM-ECG Browser

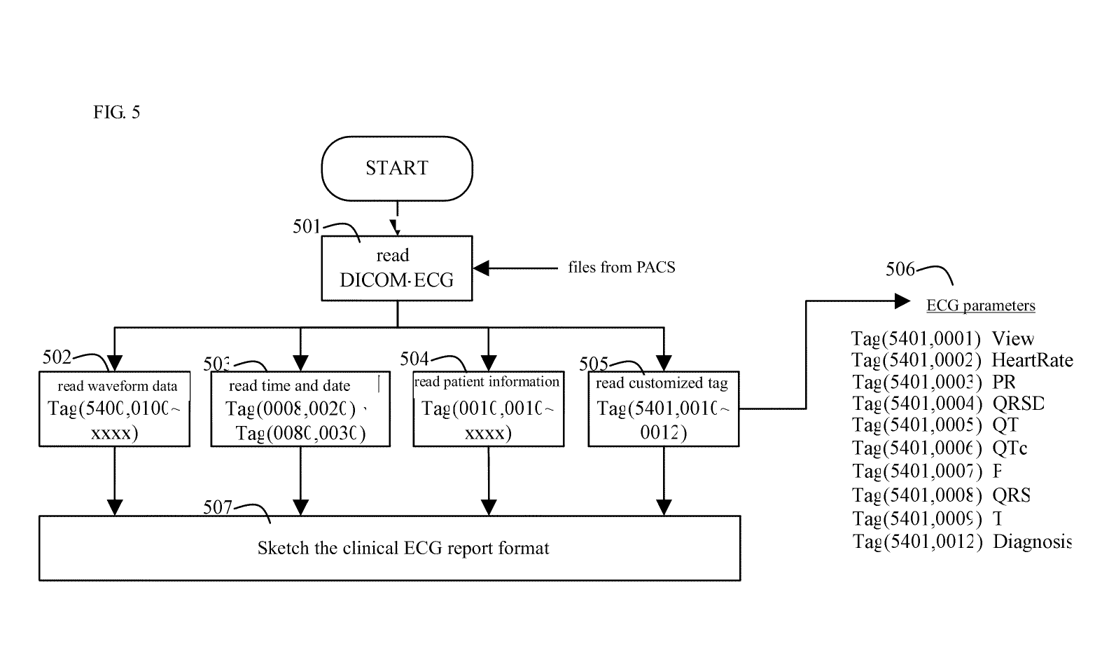

As shown in FIG. 5, the 12-Lead ECG report could be viewed via a DICOM-ECG browser from the constructed DICOM-ECG files. The preferred embodiments were described as follows.

First, DICOM-ECG file 501 was read; then the required tags were read, which were, respectively, the Chinese text part of the customized tags 505-506, patient information 504 and the time and date of examination 503. The retrieved ECG signal tags and waveform data together formed the display of the 12-Lead ECG diagnosis report 507.

### Example 5

### Flow Charts of DICOM-ECG, HIS and PACS

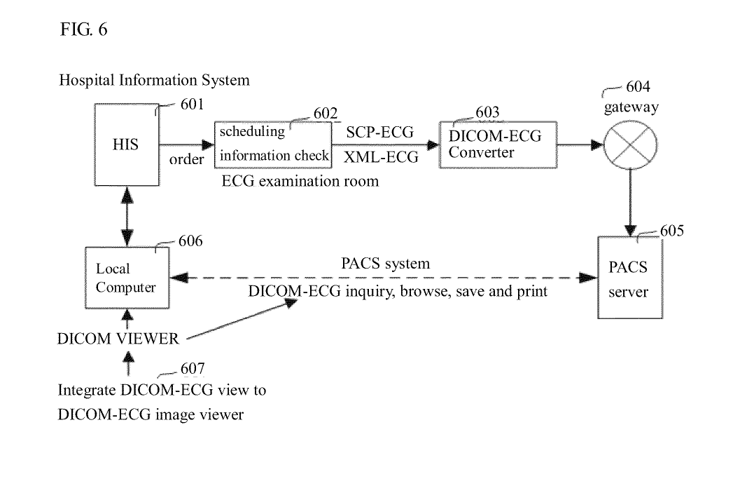

As shown in FIG. 6, the scheduling information check 602 was processed via HIS 601 in the ECG examination room, while the SCP-ECG or XML-ECG was converted to DICOM-ECG 603. Through the gateway 604, the files were stored in PACS 605 and the completion of the check was reported to HIS 606 for the purpose of bilateral communication between the medical personnel and the browser 607.

### Example 6

### The Browsing Interface of the DICOM-ECG Browser

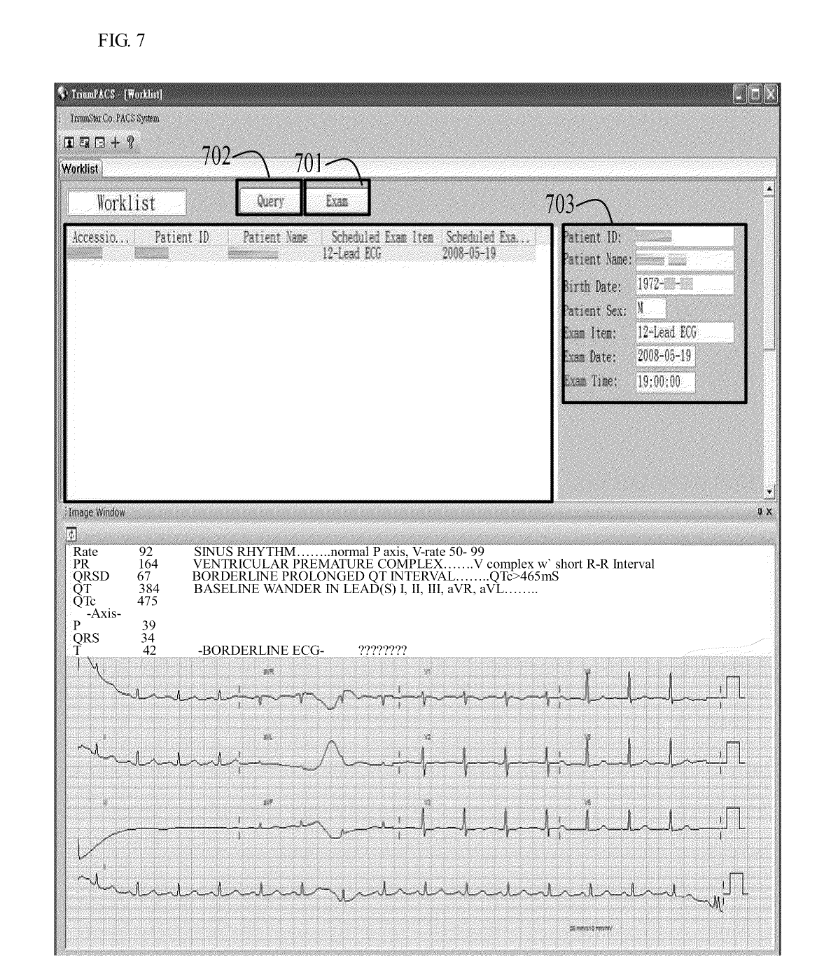

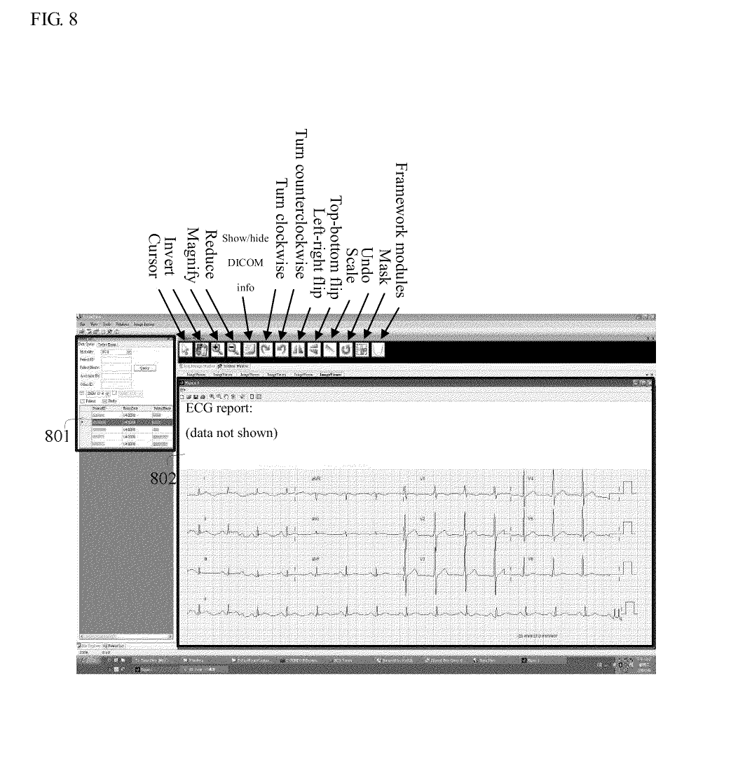

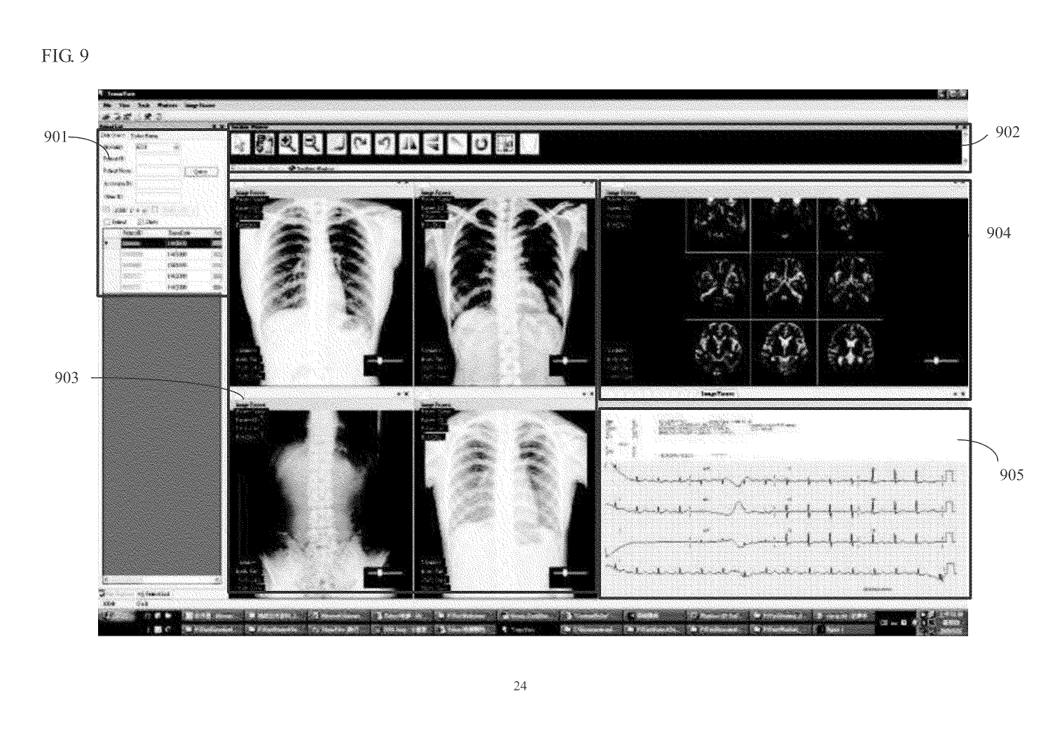

As shown in FIG. 7, the ECG examination room arranged the ECG examination schedule through the 12-Lead ECG examination scheduling information system. Using this system interface 702, operators could confirm the identity 703 of the patient heading to the ECG room and sent signals back to the HIS 701 after the completion of examination. After the 12-Lead ECG files were processed with the method mentioned above, subjected to noise reduction and then constructed to a DICOM-ECG 12-lead ECG report, the present invention provided a DICOM-ECG browser to display the converted files above as shown in FIG. 8. For the convenience of the intranet in the hospital, the ECG browser was constructed and integrated into the PACS system, enabling users to make inquiries for specific ECG file information 801, as shown on the left, and browse the ECG report 802, as shown on the right. It not only showed the DICOM 12-Lead ECG, but also allowed users to use functions, such as indicator, reverse, zoom in, zoom out, show/hide DICOM information, turn clockwise and counterclockwise, left-right and top-bottom flips, as well as features such as the scale, undo function, masks and framework modules, to show the original image objects. The present invention integrated the DICOM-ECG browser with patient list 901 and toolbox windows 902 into other medical image browsers, so simultaneous diagnosis with other medical images like the x-ray photographs 903, the MRI images, the CT images 904 and the ECG reports 905 can be achieved while browsing the ECG, as shown in FIG. 9.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A data processing system of 12-Lead DICOM ECG that integrates with HIS or PACS, comprising:
(a) a decoding and signal processing device that automatically retrieves SCP-ECG or XML-ECG used in a clinical ECG instrument;
(b) a device which converts the decoded ECG signals to DICOM-ECG;
(c) an ECG processing unit of 12-Lead DICOM ECG information management system, which receives signals from HIS or transmits DICOM-ECG to PACS; and
(d) a 12-Lead DICOM-ECG browser that is switched on using HIS or PACS.

2. The system of claim 1, wherein the decoding device of SCP-ECG comprises:
(a) a device for processing the decoding of sections 0, 1, 8, 128; and
(b) a device for processing Huffman decoding.

3. The system of claim 1, wherein the decoding device of XML-ECG comprises:
(a) a device for reading report information (reportinfo tag), data acquisition information (measurements tag), patients' information (patient tag), interpretation information (interpretations tag) and waveforms information (waveforms tag); and
(b) a signal-processing device.

4. The system of claim 2, wherein obtained data from the decoding device are transformed into ECG signals by stationary wavelet conversion.

5. The system of claim 4, wherein the ECG signals comprise raw data of ECG waveforms or ECG diagnostic information.

6. The system of claim 4, wherein the ECG diagnostic information comprises an accession number, name, gender, date of examination, time of examination, or ECG diagnosis result of individual patients.

7. The system of claim 3, wherein obtained data from the decoding device are transformed into ECG signals by stationary wavelet conversion.

8. The system of claim 7, wherein the ECG signals comprise raw data of ECG waveforms or ECG diagnostic information.

9. The system of claim 7, wherein the ECG diagnostic information comprises an accession number, name, gender, date of examination, time of examination, or ECG diagnosis result of individual patients.

10. The system of claim 1, wherein the device which converts the decoded ECG signals to DICOM-ECG further comprises generation of a core file and an index file.

11. The system of claim 10, wherein the core files are subdivided into the format conversion of SCP-ECG core files and XML-ECG core files.

12. The system of claim 11, wherein the format conversion of SCP-ECG core files sequentially comprises signal length extension, sequence order swapping, lead order swapping, signal length calculation, and the writing of waveform lengths, level lengths or waveform data.

13. The system of claim 11, wherein the format conversion of XML-ECG core files sequentially includes lead order swapping, signal length calculation, and the writing of waveform lengths, level lengths or waveform data.

14. The system of claim 10, wherein the index file utilizes the ECG diagnosis information to write custom tags, patient information or retrieves time and date.

15. The system of claim 1, wherein the ECG processing unit of 12-Lead DICOM ECG information management system is a device which stores files in PACS and reports completion of diagnosis and communication with the information page to the HIS

16. The system of claim 15, wherein the PACS is used to integrate DICOM files in medical institutes.

17. The system of claim 1, wherein the DICOM-ECG browser is linked to PACS or HIS system for remote operation.

18. The system of claim 17, wherein the operation comprises creating new files, opening old files, saving files, printing, magnifying, shrinking or moving the files.
